(19) United States
(12) Patent Application Publication
(10) Pub. No.: US 2012/0064551 A1
(43) Pub. Date: Mar. 15, 2012

(54) METHOD FOR THE PREOPERATIVE DETERMINATION OF THE INTRAOPERATIVE RISK OF BLEEDING OF A PATIENT

(76) Inventor: Wolfgang Korte, St. Gallen (CH)

(21) Appl. No.: 13/055,640

(22) PCT Filed: Jul. 23, 2009
(86) PCT No.: PCT/CH2009/000262

§ 371 Date: Dec. 2, 2011

(30) Foreign Application Priority Data
Jul. 24, 2008 (CH) 01157/08

(51) Int. Cl. C12Q 1/56

(52) U.S. Cl. 435/13

(57) ABSTRACT

In order to determine the intraoperative risk of bleeding preoperatively, both the content of fibrin monomer (FM) and the partial thromboplastin time (PTT) are determined in a blood or plasma sample.

### CROSS-REFERENCE TO RELATED APPLICATIONS

This application claims priority to PCT Patent Application No. PCT/CH20091000262 filed on Jul. 23, 2009 and Swiss Patent Application No. 01157/08 filed on Jul. 24, 2008, the entirety of each of which is incorporated by this reference.

### BACKGROUND

1. Field of the Invention

The invention relates to a method for preoperative determination of a patient's risk regarding tendency toward intraoperative hemorrhage.

2. State of the Art

In surgical operations, complications can occur due to intraoperative hemorrhages. If diffuse hemorrhages occur in the wound region or at the wound edges, without any evident mechanical cause being present, then it must be assumed that there is a tendency toward hemorrhage due to coagulopathy.

Various tests are already known for clarifying hemorrhages or coagulation problems. One frequently used test is known as the PTT or aPTT test ([in English:] activated partial thromboplastin time). This test measures the time span (in seconds) that is required for formation of a blood clot in a test tube. For this purpose, a citrate blood sample is centrifuged and the supernatant plasma is used.

Another test is the PT test ([in English:] prothrombin time), which is referred to as a Quick test when carried out as a ratio with regard to a normal plasma.

The aPTT and PT tests have also already been used, in the past, for determining the risk of a tendency toward intraoperative hemorrhage. However, various studies have shown that these tests are only poorly suited for this purpose. For example, in an article published in 2005 ([in English:] “Preoperative fibrin_{—} monomer measurement allows risk stratification for high intraoperative blood lective [sic] surgery” in THROMBOSIS & HAEMOSTASIS, July 2005, Vol. 1, pages [sic] 211), it was found that intraoperative blood loss has no relationship with the prothrombin time ([in English:] prothrombin time), the aPTT values, and the blood platelet count.

In an article authored by Teruya et al., with the title “[in English:] A normal aPTT does not guarantee adequate coagulation factor levels” (in ANESTHESIOLOGY, Vol. 94, No. 3, March 2001, page 542), it is explained that a normal PTT does not predict that normal coagulation factor activity also exists. It is found that the aPTT cannot be used as a predictive factor for a lowered coagulation factor level, but rather, on the contrary, it is unreliable. Therefore the use of aPTT preoperatively cannot be recommended. Also, in the article mentioned, no relationship between aPTT and the tendency toward intraoperative hemorrhage, in particular, is produced.

Patients with an elevated tendency toward intraoperative hemorrhage demonstrate elevated coagulation activation. The value of the aPTT is influenced, among other things, also by the extent of the coagulation activation ([in English:] Ten Boekel E, Bartels P. Abnormally short activated partial thromboplastin times are related to elevated plasma levels of TAT, F1+2, D-dimer and FVIII:C. Pathophysiol Haemost Thromb. 2002; 32: 137-142).

It is known that fibrin monomer (FM) can serve for determining a “prethrombotic state,” in other words for early detection and monitoring of coagulation-activating processes; this occurs, for example, in the case of deep vein thrombosis (DVT) or disseminated intravascular coagulation (DIC). However, fibrin monomer (FM) is also suitable for detecting an elevated risk of intraoperative hemorrhages. For example, the use of fibrin monomer as a diagnostic marker for a patient's risk of intraoperative hemorrhages is described in DE 198 33 844. It was found that fibrin monomers are best suited for assessing the risk of a tendency toward intraoperative hemorrhage, among the potential markers investigated.

Accordingly, the fibrin monomer determination (FM) has already proven itself as a preoperative screening for assessing the tendency toward intraoperative hemorrhage, within the scope of clinical studies.

However, there continues to be a need to improve the predictive quality of the test. It is therefore the task of the present invention to further improve the preoperative screening that has been described.

### SUMMARY OF THE INVENTION

As has been mentioned above, the aPTT is partly dependent on the extent of the coagulation activation, but cannot be used in isolation for risk stratification of a tendency toward intraoperative hemorrhage. On the other hand, the fibrin monomer determination already shows good potential for preoperative risk stratification of a tendency toward intraoperative hemorrhage.

The task is accomplished in that the two study methods are combined, and in a sample of the patient, both the content of fibrin monomer (FM) and the partial thromboplastin time (PTT) are determined and put into relation with one another. In comparison with the known method, in which only the content of fibrin monomer (FM) is checked, the method according to the invention leads to an increased diagnostic sensitivity, in other words the ability to determine the actual risk patients. At the same time, the specificity also increases; the improvement of an assay, leading to an improvement of both sensitivity and specificity, is a goal that should be aimed at but is unfortunately rarely achieved. The solution of the combination of aPTT and fibrin monomer presented here allows achieving this goal.

It is advantageous if fibrin monomer (FM) is determined immunologically. Citrate plasma may be used as a sample for the determination of the content of fibrin monomer, and also for the determination of the activated thromboplastin time (aPTT).

The invention therefore also relates to the use of fibrin monomer (FM) in combination with the activated partial thromboplastin time (aPTT) as a diagnostic marker in the assessment of the risk of a tendency toward intraoperative hemorrhage.

### BRIEF DESCRIPTION OF THE DRAWINGS

FIG. 1 shows an ROC curve.

### DETAILED DESCRIPTION OF THE INVENTION

### EXAMPLE 1

### Known Fibrin Monomer Determination as Preoperative Screening for Exclusion of a Tendency Toward Intraoperative Hemorrhage

In 226 consecutive patients with various operations, without extracorporeal circulation and with artery probe, the preoperative fibrin monomer concentration (FM) was investigated prospectively, and correlated with the occurrence of an intraoperative disorder of hemostasis (IDH). The patient group was already described in Korte et al., Clin. Chem. Lab. Med. 1998, 36 (4), 235-240.

Sample-taking took place, after rejection of the first 3 ml, from arterial probes flushed with 0.9% NaCI solution, in 0.125 M Na citrate (9+1). IDH was defined, in this connection, as the occurrence of diffuse hemorrhages in the wound region or at the wound edges, without any evident mechanical cause, after adequate local hemostasis had already been achieved.

Fibrin monomer was determined using the Enzymun-Test® FM using an ES-300 device.

| | FM | FM | D-dimer tinaquant | | | |
|---|---|---|---|---|---|---|
| | (μg/ml) | (μg/ml) | (ng/ml) | | D-dimer latex (mg/l) | |
| | 75^{th} | 90^{th} | 75^{th} | 90^{th} | 75^{th} | 90^{th} |
| | percentile | percentile | percentile | percentile | percentile | percentile |
| Percentile | 14.50 | 40.50 | 0.97 | 2.30 | 0.75 | 1.50 |
| of | | | | | | |
| patients | | | | | | |
| without | | | | | | |
| IDH | | | | | | |
| RR¹ for | 3.79 | 3.16 | 1.56 | 2.56 | 2.88 | 2.21 |
| IDH | | | | | | |
| OR² | 4.44 | 3.77 | 1.64 | 2.94 | 3.29 | 2.46 |
| Sensitivity | 60% | 30% | 35% | 25% | 45% | 20% |
| Specificity | 75% | 90% | 75% | 90% | 80% | 91% |
| Pos. | 19% | 22% | 12% | 19% | 18% | 17% |
| predictive | | | | | | |
| value | | | | | | |
| Neg. | 95% | 96% | 92% | 92% | 94% | 92% |
| predictive | | | | | | |
| value | | | | | | |

| | Cut-off FM (μg/ml) | | | | | | | | |
|---|---|---|---|---|---|---|---|---|---|
| | 12 | 11 | 10 | 9 | 8 | 7 | 6 | 5 | 4 |
| Value > | 12/20: | 12/20: | 13/20: | 14/20: | 14/20: | 14/20: | 15/20: | 17/20: | 18/20: |
| cut-off | 63/206 | 66/206 | 71/206 | 78/206 | 88/206 | 101/206 | 110/206 | 121/206 | 137/206 |
| in patients | | | | | | | | | |
| IDH vs. | | | | | | | | | |
| non-IDH | | | | | | | | | |
| Sensitivity | 60 | 60 | 65 | 70 | 70 | 70 | 75 | 85 | 90 |
| (%) | | | | | | | | | |
| Specificity | 69 | 68 | 65 | 62 | 57 | 51 | 47 | 41 | 33 |
| (%) | | | | | | | | | |
| Pos. | 16 | 15 | 15 | 15 | 14 | 12 | 12 | 12 | 12 |
| predictive | | | | | | | | | |
| value (%) | | | | | | | | | |
| Neg. | 95 | 95 | 95 | 95 | 95 | 95 | 95 | 97 | 97 |
| predictive | | | | | | | | | |
| value (%) | | | | | | | | | |

¹RR: relative risk
²OR: odds ratio

### EXAMPLE 2

### Combination of Preoperative aPTT and Fibrin Monomer Determination for Improved Exclusion of a Tendency Toward Intraoperative Hemorrhage

In a second approach, from the population described above, in the case of 154 patients who could be evaluated, not only the FM but also the aPTT was determined, with Pathromtin SL on a BCS device. FM and aPTT were both determined using the batch method, from aliquots of the samples stored at −80° C., and a ratio of the measurement values was formed. Furthermore, in the case of these patients, the 75^{th} percentile of the intraoperative blood loss was calculated; this was 500 ml. The preoperative use of the aPTT together with the FM values, in a ratio, allows a prediction of an intraoperative blood loss of more than 500 ml, in other words above the 75^{th} percentile, at the optimal point, according to the ROC curve (10.7252), with a sensitivity of 94.44%, a specificity of 52.68%, a positive predictive value of 39.1%, and a negative predictive value of 96.7%.

In order to determine the optimal point, sensitivity, specificity, positive (PPV) and negative predictive value (NPV) were investigated at different values of the ratio between aPTT and FM (“criterion”), and presented in Table 1. For the sake of a better overview, the points that yielded a sensitivity of 80% to 100% were shown.

From this, it is evident that the value of 10.7252 represents the most optimal point if sensitivity, specificity, positive and negative predictive value are taken into consideration. It is also evident that by means of displacement of the point, above all a greater sensitivity, i.e. a higher negative predictive value (up to 100%) can be achieved.

TABLE 1

| Criterion | Sensitivity | Specificity | PPV | NPV |
|---|---|---|---|---|
| <=9.0515 | 80.56 | 58.04 | 38.2 | 90.3 |
| <=9.3871 | 83.33 | 58.04 | 39.0 | 91.5 |
| <=9.4041 | 83.33 | 57.14 | 38.5 | 91.4 |
| <=9.4921 | 83.33 | 56.25 | 38.0 | 91.3 |
| <=9.5074 | 86.11 | 56.25 | 38.7 | 92.6 |
| <=9.7361 | 86.11 | 55.36 | 38.3 | 92.5 |
| <=9.7561 | 86.11 | 54.46 | 37.8 | 92.4 |
| <=9.7619 | 86.11 | 53.57 | 37.3 | 92.3 |
| <=9.8095 | 88.89 | 53.57 | 38.1 | 93.7 |
| <=10.1905 | 91.67 | 53.57 | 38.8 | 95.2 |
| <=10.7059 | 91.67 | 52.68 | 38.4 | 95.2 |
| <=10.7252* | 94.44 | 52.68 | 39.1 | 96.7 |
| <=10.8127 | 94.44 | 51.79 | 38.6 | 96.7 |
| <=10.8257 | 94.44 | 50.89 | 38.2 | 96.6 |
| <=10.8609 | 94.44 | 50.00 | 37.8 | 96.6 |
| <=11.0744 | 94.44 | 49.11 | 37.4 | 96.5 |
| <=11.3704 | 97.22 | 49.11 | 38.0 | 98.2 |
| <=11.8321 | 97.22 | 48.21 | 37.6 | 98.2 |
| <=11.9929 | 97.22 | 47.32 | 37.2 | 98.1 |
| <=12.1254 | 97.22 | 46.43 | 36.8 | 98.1 |
| <=12.439 | 97.22 | 45.54 | 36.5 | 98.1 |
| <=12.4701 | 100.00 | 45.54 | 37.1 | 100.0 |

The corresponding values for the sole use of the FM value yield a sensitivity of 91.67%, a specificity of 51.75%, a positive predictive value of 37.5%, and a negative predictive value of 95.2%.

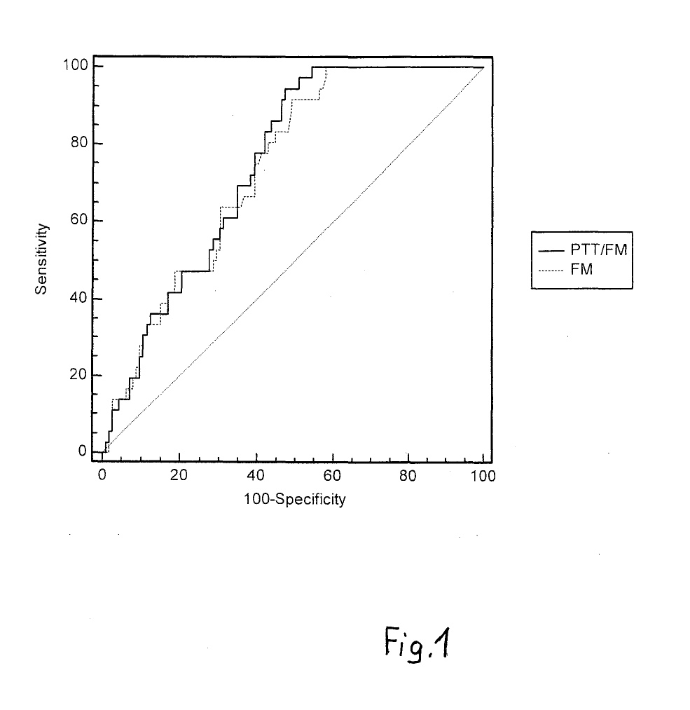

FIG. 1 shows an ROC curve with the values for fibrin monomer (FM) alone and the values for the combination of partial thromboplastin time (PTT) and fibrin monomer (FM).

Receiver operating characteristic (ROC) or ROC curves serve, in medicine, for the evaluation of diagnostic tests (Ulrich Abel: Bewertung diagnostischer Tests [Evaluation of diagnostic tests], Hippokrates Verlag [publishing company], Stuttgart 1993). The sensitivity represents the ability of a diagnostic method to identify the actual risk patients. The specificity represents the ability of a diagnostic method to avoid false positive tests. From FIG. 1, it is now evident that the combination of a PTT test and an FM test delivers better results than an FM test alone, in other words if the measurement results are put into relation with one another.

1. A method for pre-operative determination of a patient's risk regarding tendency toward intraoperative hemorrhage, comprising:
providing a sample of the a patient;
determining both a content of fibrin monomer and a partial thromboplastin time;, and
putting the content of fibrin monomer and the partial thromboplastin time into relation with one another.

2. The method according to claim 1, further comprising determining the fibrin monomer immunologically.

3. The method according to claim 1, further comprising providing the sample as a blood sample.

4. The method according to claim 1, further comprising providing the sample as a plasma sample.

5. The method of claim 1, further comprising using the determination of the fibrin monomer in combination with the thromboplastin time as a diagnostic marker in the estimation of a risk of a tendency toward an intraoperative hemorrhage of the patient.
